(19) United States
(12) Patent Application Publication
(10) Pub. No.: US 2022/0257178 A1
(43) Pub. Date: Aug. 18, 2022

(54) APPARATUS FOR DETERMINING INDICATION OF PAIN

(71) Applicant: KIPUWEX OY, Oulu (FI)

(72) Inventor: Marko HÖYNÄLÄ, Oulu (FI)

(21) Appl. No.: 17/624,677

(22) PCT Filed: Jul. 8, 2020
(86) PCT No.: PCT/FI2020/050489

§ 371 Date: Jan. 4, 2022

(30) Foreign Application Priority Data
Jul. 10, 2019 (FI) 20195626

(51) Int. Cl. A61B 5/00; A61B 5/0205; A61B 5/021; A61B 5/024; A61B 5/08; A61B 5/0531; A61B 5/145

(52) U.S. Cl. CPC A61B 5/4824; A61B 2562/0204; A61B 5/7246; A61B 5/7267; A61B 5/7282; A61B 5/02055; A61B 5/021; A61B 5/02405; A61B 5/0816; A61B 5/0531; A61B 5/14542; A61B 5/7475; A61B 5/742; A61B 5/7405; A61B 5/0077; A61B 2560/0223; A61B 5/7289

(57) ABSTRACT

There is provided an solution for analysing pain. The solution comprises obtaining (200) video stream of a subject; obtaining (202) audio stream of the subject; obtaining (204) one or more biosignal stream of the subject; the obtained data being synchronised with each other and related to an event of care of the subject, and covering a given first time window before, during and after the event of care; selecting (206, 208) points around the event of care and within the first time window and a second time window shorter than the first time window; selecting (210) video, audio and biosignal data stream at the set of time points, the length of the data stream being the duration of the second time window; controlling (212) interface to play selected data streams; obtaining (214) using an interface from a plurality of users inputs describing estimates of pain experienced by the subject; processing (216) the obtained inputs and determining (220) a correlation between the estimated pain experienced by the subject and the audio and biosignal data streams.

### FIELD

The present invention relates to an arrangement for analysing pain. More particularly, the present invention relates to determining an indication of pain experienced by a subject.

### BACKGROUND

Health status such as pain is difficult to measure and analyse objectively. There are multiple pain scales and questionnaires and tables to estimate the pain experienced by a patient herself or nurse or any healthcare specialist. However, all current estimation solutions are subjective and somewhat unreliable. Based on the estimator, the resulted estimate of pain experienced by a subject may vary greatly.

At present there is not a suitable method for obtaining an estimate of experience pain which could be denoted as objective value.

### BRIEF DESCRIPTION

There is provided the subject matter of independent claims. Some embodiments are disclosed in dependent claims.

One or more embodiments and examples are set forth in more detail in the accompanying drawings and the description below. Other features will be apparent from the description and drawings, and from the claims.

### BRIEF DESCRIPTION OF THE DRAWINGS

In the following embodiments will be described in greater detail with reference to the attached drawings, in which

FIGS. 1A and 1B illustrate a system according to an embodiment;

FIG. 2 is a flow chart illustrating an embodiment;

FIG. 3 illustrates an example of obtained data streams in an embodiment;

FIGS. 4, 5, 6 and 7 are tables illustrating some embodiments.

### DETAILED DESCRIPTION

The following embodiments are exemplifying. Although the specification may refer to “an”, “one”, or “some” embodiment(s) in several locations of the text, this does not necessarily mean that each reference is made to the same embodiment(s), or that a particular feature only applies to a single embodiment. Single features of different embodiments may also be combined to provide other embodiments.

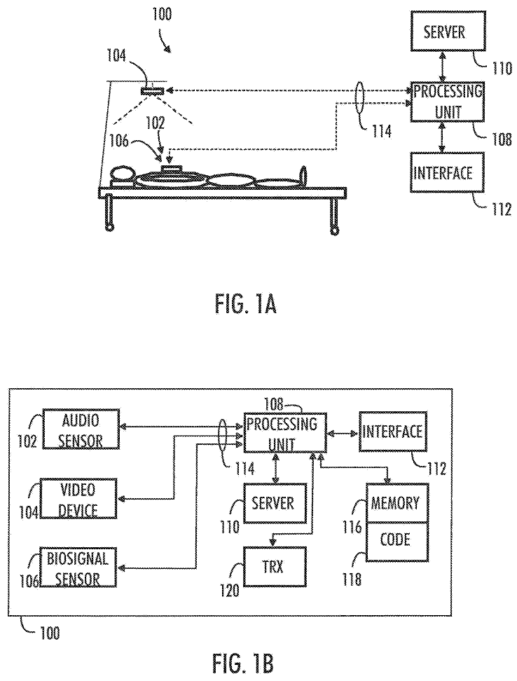

FIGS. 1A and 1B illustrate an example of a system which may be utilised in determining an indication of pain and an estimate of pain experienced by a subject or a patient. An audio sensor 102 is configured to obtain an audio data stream of the subject. Aa video capturing device 104 is configured to obtain a video data stream of a subject. One or more biosensors 106 are configured to measure one or more biosignals of a subject.

A processing unit 108 communicatively coupled 114 with the audio sensor 102, video capturing device 104 and the one or more biosensors 106, is configured to control the operations of the sensors and devices.

The system may further comprise a server 110 or data base configured to store data and user interface 112 for displaying data and obtaining input. The audio sensor 102 may be any kind of audio sensor, such as a microphone, configured to convert sound into an electrical signal. That is, the audio sensor 102 may convert an audio input into an audio signal (i.e. electrical audio signal). The audio sensor may comprise one or more audio transducers. For example, to measure intensity of sound, it may be beneficial to use more than one microphone.

The video capturing device 104 may be any kind of video recorder capable of recording video, typically, but not necessarily to a suitable digital video format. Any suitable digital video format may be used. The device can be attached to a rack or stand so that camera monitor see the subject, for example subject's face and upper body. The device can be attached to the inner roof of the room where the subject is. The captured video can be continuous video stream or 1-10 sec video clips covering the desired time. Video may comprise both video image and an audio soundtrack.

In an embodiment, in the field of view of the device 104 are also the audio sensor 102 and one or more biosignal sensors 106 and possible indicators (LEDs) on them. The device 104 may also detect sound signals from those sources. The device may comprise wireless transceiver such as Bluetooth®, WiFi or a cellular transceiver or capability for wired combinations such as Ethernet or USB (Universal Serial Bus).

In an embodiment, the device may be realised with a user terminal such as a smartphone or a tablet computer, for example. The user terminal may comprise an audio input and video recording capabilities.

The biosensor 106 is configured to measure one or more biosignals of a subject 114. The subject may refer to, for example, a person or a human. For example, the subject may be a patient, such as a patient in a hospital. The biosensor 106 may have a measurement head or heads configured to be placed in contact with a body tissue of the subject. The sensor may be a wearable device attached on the subject's wrist with a strap or patient's skin on a chest with a strap or a sticker. The biosensor 106 may be configured to measure one or more biosignals of the subject. Biosignals may comprise, but are not necessarily limited to, Heart Rate Variability (HRV), heart rate, respiration rate, blood oxygen level, temperature, and blood pressure. Measuring such biosignals is generally known from the art of biosignal measuring. The biosensor 106 may measure said biosignals and provide raw measurement data and/or processed measurement data as an output. For example, the biosensor 106 may pre-process the raw measurement data and provide pre-processed measurement data as an output. Pre-processing may comprise, for example, filtering, modulating, demodulating and/or converting (e.g. analog-to-digital converting) the detected biosignal or biosignals before outputting the pre-processed biosignal data. However, in some embodiments, the processing unit 108 receives real-time audio data and biosignal data from the respective sensors, and process the data in real-time as described below. Naturally, there may be some delay caused by, for example, non-ideal transmission link(s). Biosensor 106 may comprise one or more sensors, such as optical heart activity sensor, electrode(s) (i.e. electrode based measurements of heart rate and/or respiration rate), temperature sensor, blood pressure sensor, blood oxygen level sensor. Hence, one or more biosignals of a subject may be measured. The sensor may detect sound by a microphone, heart rate with an optical plethysmograph, temperature with a temperature sensor, breathing with a chest band resistive, capacitive or inductive sensor, ECG (electrocardiogram) with electrodes, EMG (electromyografia) with electrodes, for example. As described such sensors are generally known in the art of measuring biosignals and will not be disclosed in further detail.

In an embodiment, the biosensor 106 can be also a noncontact sensor such as a RF radar to measure movements of body or chest of the subject to determine heart rate. The biosensor 106 can be also a noncontact sensor such as a camera or IR sensor to measure temperature of subject skin. By detecting a variation of the temperature changes the heart rate can be determined. This kind of camera detector can be realized with the video capturing sensor/device.

In an embodiment, the video stream provided by the video capturing device 104 may be processed such that heart rate and skin temperature may be obtained from the video stream.

The system 100 further comprises a processing unit 108. The processing unit 108 may comprise one or more processors coupled with one or more memories 116, the one or more memories 116 comprising program code 118, wherein the program code 118 may cause the one or more processors to execute functions of the processing unit 108. In another example, the processing unit 108 comprises one or more circuitries configured to perform the functions of the processing unit 108. In another example, the processing unit 108 comprises both processor(s) controlled at least partially by the program code, and dedicated circuitry or circuitries executing a pre-configured functionality. Such dedicated circuitries may include, for example, Field-Programmable Gate Array (FPGA) and/or Application Specific Integrated Circuitry (ASIC) circuitries. The processing unit 108 is also operatively connected to server 110.

The processing unit 108 may be communicatively coupled 114 with the audio sensor 102, video capturing device and the biosensor 106. Said coupling may be established using wired and/or wireless communication. For the communication, the processing unit 108 may utilize a communication circuitry 120 (shown as TRX 120 in FIG. 1B). The communication may be one-directional (e.g. receiving data from the sensors by the processing unit 108) or bidirectional (e.g. receiving data from sensor and possibly configuring the sensors). TRX 120 may not be necessary if the sensors and/or device 102, 104, 106 are connected to the processing unit via conductive traces (e.g. wires). However, TRX 120 may utilized to enable use of one or more communication protocols and/or interfaces, such as Local Area Network (LAN), Universal Serial Bus (USB), Bluetooth (e.g. Bluetooth smart), Wireless LAN (WLAN), infrared, and/or cellular communication (e.g. 2G, 3G, 4G, 5G). For example, the TRX 120 may enable communication on industrial, scientific and medical (ISM) radio bands according to one or more communication protocols. In an embodiment, some or all connections between different units of the system 100 may be realised with Internet Protocol, IP, connections utilising normal protocols known in the art.

The processing unit 108 may be configured to obtain audio data from the audio sensor 102, video capturing device 104 and biosignal data from the biosensor 106. The audio data may carry and/or comprise information about the detected audio signal, the video data may carry and/or comprise information about the captured video signal and the biosignal data may carry and/or comprise information about the detected biosignal(s). The audio data, video data and biosignal data may be received directly from the respective sensors/device or it may be stored to the memory 118 ore server 110, wherein the processing unit 110 may obtain (e.g. fetch) the data from the memory or server.

In an embodiment, the processing unit 108 can be configured to control and set parameters of audio sensor, video device and biosignal sensor. For example it can modify measuring parameters such as sampling frequency, to measuring time, signal bandwidth, and various limits. In an embodiment, the processing unit can also set filters, and control zoom or optics or even direct the sensing beam of the audio sensor or video device.

The audio data, video data and the biosignal data may be time-synced (i.e. time-synchronized) with each other. This may mean that the audio and video data and the biosignal data represent measurements from the same measurement period and different samples in the audio data timely correspond to different samples in the biosignal data. For example, if a first audio signal and first video signal are measured at a first time instant, the biosignal data may comprise a first biosignal sample measured at said first time instant. Hence, the first audio signal, and the first video signal may timely correspond to the first biosignal sample. Similarly, for example, if a second audio signal and a second video signal are measured at a second time instant (e.g. being different than the first time instant), the biosignal data may comprise a second biosignal sample measured at said second time instant. Hence, the second audio signal and the second video signal may timely correspond to the second biosignal sample.

It needs to be understood that there may be a plurality of different samples over a certain time period, for example. So, the measurement of sound or video may be performed simultaneously with the measurement of the biosignals(s). It needs to be noted that even though the measurement would be simultaneous, in some cases, it may be possible that the audio or video data and the biosignal data is not time-synced due to, for example, delay in the system. Hence, the system (e.g. processing unit) may sync (i.e. synchronize) the audio and video data and the biosignal data if they are not initially in-sync. It is further noted that the time-synced audio and video data and biosignal data should be understood broadly to cover situations in which a certain event (e.g. measuring period or periods) at a certain time instant(s) (e.g. time period or periods) may be detected from both the audio and video data and the biosignal data. For example, measuring period may be 2-30 seconds, and audio signal and biosignal may be measured simultaneously with the accuracy of 1-1000 milliseconds. That is, the different signals may be measured for said measuring period, and their correspondence with each other may be within said accuracy limit.

In an embodiment, system comprises a user interface 112. The user interface 112 may comprise a keyboard, virtual keys, a voice control circuitry, a display element(s) (e.g. display and/or indication lights) and/or a speaker. In an embodiment, the user interface is realised with a user terminal such as a smartphone or a tablet computer, for example.

In an embodiment, the processing unit 108 and the user interface 112 are integrated to one apparatus. The apparatus may be realised with a user terminal such as a smartphone or a tablet computer, for example.

In an embodiment, the system 100 comprises a server 110 computer or server computers configured to store audio, video and biosignal data. The server computer may comprise a controller. In an embodiment, the server may be located in a cloud service and be accessible via the Internet.

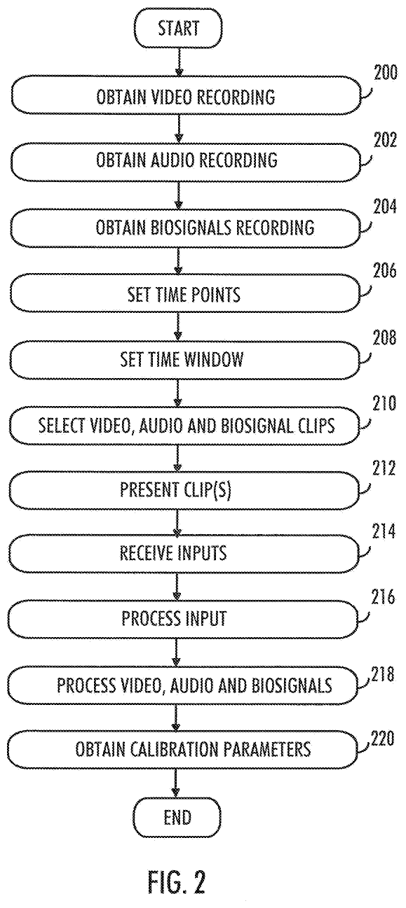

FIG. 2 shows a flow chart illustrating an embodiment of determining an indication of pain.

In the embodiment of FIG. 2, an event of care related to a subject such as a patient is monitored. The event of care can be a care operation, cleaning and disinfecting a wound, vaccination, for example and it typically causes some pain to the patient.

A given first time window around the event of care is selected, during which time window measurements are made. The time window may cover time before, during and after the event of care. As a numeric non-limiting example, the first time windows starts five minutes before the event of care starts and continues till ten minutes after the event.

In step 200, video data stream of the subject is obtained utilising the video capturing device 104;

In step 202, audio data stream of the subject is obtained utilising the audio sensor 102

In step 204, biosignal data stream of the subject is obtained utilising the one or more biosignal sensor 106.

In an embodiment, the processing unit may control the video recording device, audio and biosignal sensors. The video, audio and biosignal data are synchronised with each other, related to an event of care of the subject, and cover the first time window before, during and after the event of care.

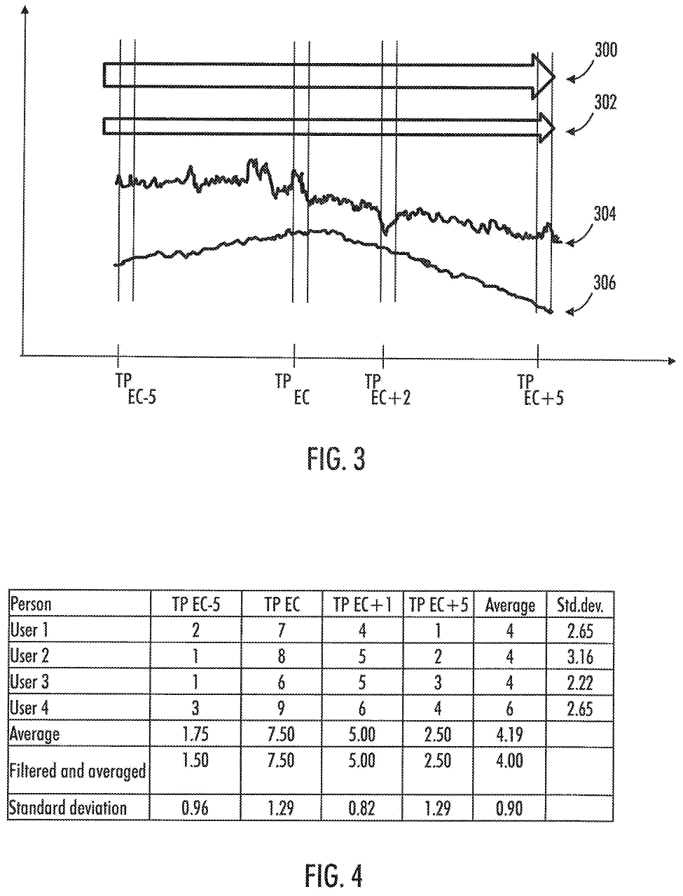

FIG. 3 illustrates an example of obtained data streams during the first time window. In the example of FIG. 3, the data streams comprise video stream 300, audio stream 302, first biosignal (heart rate) 304 and second biosignal (temperature) 306.

The obtained data streams may be stored in the server 110.

In step 206, a set of timepoints around the timepoint of the event of care are selected. In this example there are four time points, where the time to points are during the event, TP_{EV}, 5 minutes before the event, TP_{EV-5}, and 2 and 5 minutes after the event, TP_{EV+2}, TP_{EV+5}.

In step 208, a second time window is selected, the second time window being shorter than the first time window and used starting from each selected timepoint. The time window is, for example, 30 seconds.

In step 210, clips of video, audio and biosignal data streams at each time point of the set of time points are selected, the length of the data stream clips being the duration of the second time window. In the example, of FIG. 3, clips having the length of 30 seconds are selected at the four time points TP_{EV}, TP_{EV-5}, TP_{EV+2} and TP_{EV+5}.

In step 212, the selected video clip or clips are loaded to the interface 112 and displayed. In an embodiment, the video clips are shown on the display of a user terminal or tablet computer. In an embodiment, also audio data stream clip and/or biosignal clip are played using the interface.

In step 214, the interface is configured to obtain from a plurality of users inputs describing estimates of pain experienced by the subject at the set of time points.

Thus in an embodiment, a plurality of users, such as the subject (patient), a nurse or other people may to define the pain level they assume the subject has been experienced at each set of time points during the second time window, based on the presented video. Optionally biosignal data and audio clips are shown as well.

In the example of FIG. 3, there are four time points, each being 30 seconds. Each user will give one pain value to each period, thus 4 values in total per user are received. If there are 4 analyzers/users, then there are 4 pain values to each time point totally 16 pain values.

In step 216, the processing unit is configured to process the obtained inputs, for example statistically. For example, the inputs may be averaged. In this example, if there are four users, 4 values corresponding to a time point is received. An average value for each time point may be calculated. Further, an average over the four time points may be calculated to obtain a pain estimate for the event of care.

In step 218, the processing unit is configured to, based on the inputs, determine a correlation between the estimated pain experienced by the subject and the audio and biosignal data streams.

In step 220, the processing unit is configured to store the determined correlation.

In an embodiment, the processing unit is configured to process the data of biosignal and sound around the time points (during second time window). The processing can mean averaging, filtering and other known operations and combining of data.

In an embodiment, the processing unit is configured to scale the selected audio and biosignal data clips at the set of time points to a given scale and utilise the scaled data streams when determining the correlation.

FIG. 4 is a table illustrating an example how four different users, User 1, User 2, User 3 and User 4, evaluated and estimated pain values at four different time points TP_{EV}, TP_{EV-5}, TP_{EV+2} and TP_{EV+5}.

The processing unit may be configured to calculated average or filtered and averaged values, which can be used as a selected pain value. In this example, the filtered value is achieved by removing highest and lowest score then averaging the values left. For example, in FIG. 4, the average value for TP_{EV} is 1.75 and the filtered average 1.50.

In an embodiment, the processing unit may be configured to determine an average value of the inputs given by each user. For example, in FIG. 4, the average input value of User 1 is 2.65 while the corresponding average value of User 2 is 3.16.

In an embodiment, the processing unit may be configured to calculate a scaling factor for the input given by each user based on the determined average value and scale the input of given by each user of the plurality of users with the scaling factor. Thus any possible personal bias of a user may be averaged out. In an embodiment, an identification of each user may be received to differentiate user inputs of different users. The personal bias of a user can be used when the same user is giving estimated values at another time instant.

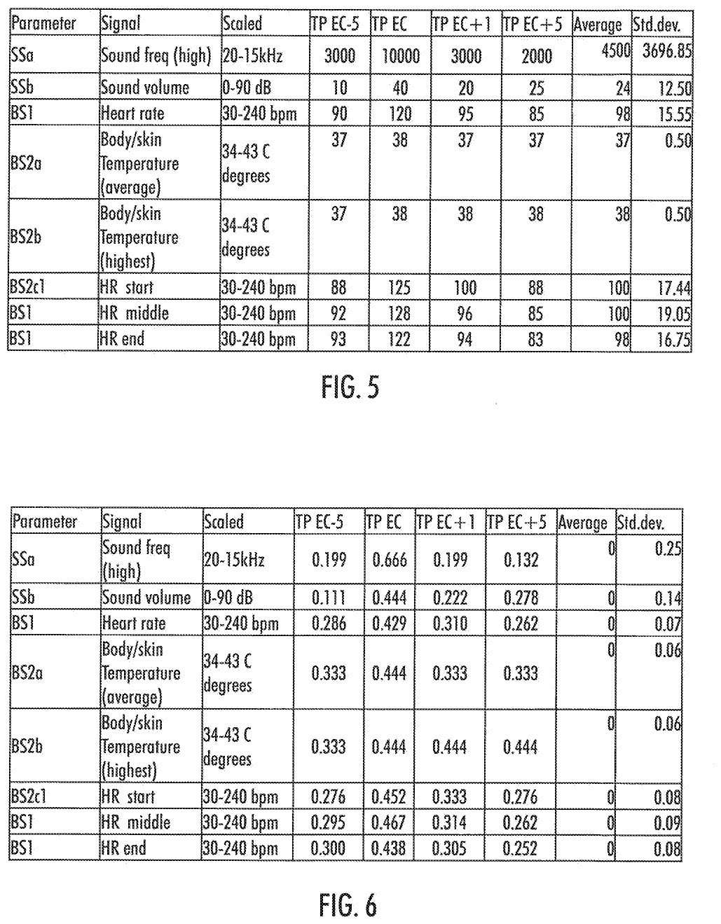

FIG. 5 is a table illustrating an example of parameter values measured during each time point TP_{EV}, TP_{EV-5}, TP_{EV+2} and TP_{EV+5}. Here SSa denotes audio frequency, SSb audio volume, biosignal BS1 heart rate, biosignal BS2a average body or skin temperature, biosignal BS2b maximum body or skin temperature, biosignal BS2c1 heart rate at start, biosignal BS2c2 heart rate at middle and biosignal BS2c3 heart rate at end.

FIG. 6 is a table illustrating an example of the same measured parameter values as in FIG. 5, but the values have been linearly scaled to be between 0 and 1. The values can be scaled many other ways such as using non-linear methods such as logarithmic, exponential or 2nd order (y(x)=ax²+b) or higher order (y(x)=ax^{m}+b) formula or any other known scaling function.

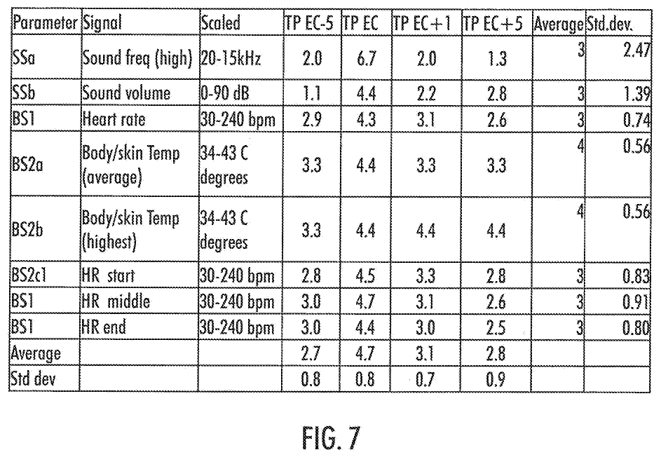

FIG. 7 is a table illustrating an example, where measured and scaled values are correlated to pain values. The each value is first scaled to be between 0-1 [0, 1] and then each value is scaled to used pain scale 0-10 [0-10]. This calculated pain value can be as an one input when calculating combination of estimation values obtained from users and measured parameters values to make a conclusion or summary pain value for each period.

For example, when changes in the audio and biosignal data correspond time wise to given user pain estimation values, such changes may be assumed to correlate with a given level of pain, for example. Various thresholds in the changes in the audio and biosignal data may be applied, for example, in determining correlations between the estimated pain experienced by the subject and the audio and biosignal data streams.

In an embodiment, measured parameters values can also be combined using more advanced mathematical methods such as a neural network to form a pain value. During the teaching phase of the neural network pain estimate values obtained from the users can be used as targeted outputs whereas parameter values can be used as input values.

The processed data and combined data values can be provided as parameters to a neural network to teach calculating a pain value for each time point. After teaching. the input data, biosignals and audio can be used to estimate the pain of the patient without further user input.

The neural network or any other similar calculation or mathematical arrangement may provide a formula to calculate a pain value based on the input data parameters as used for teaching. For example, when the formula has been created, a new data set of video, audio and biosignal may be measured and fed to the created formula and as a result a pain value for that moment may be obtained.

The neural network can be also taught so that one input parameter may be missing or not available. The formula can be used in the such case where all data inputs are not available.

The neural network can be weighted in different ways to improve the reliability and accuracy. For example the input of a more experience nurse can be weighted compared to other inputs. In addition when it is known that one user is always giving higher or lower values than in general, the inputs of this user may be lowered or heighted accordingly.

When the formula has been created it can be used as a pain indicator and it can be calculated and shown in the processing unit 108 or it can calculated in the processing unit and sent to be shown in the video device 104 which can be a user terminal or smart phone, or it can be calculated and displayed in the video device 104.

In an embodiment, the processing unit 108 carrying out the embodiments comprises a circuitry including at least one processor and at least one memory including computer program code. When activated, the circuitry causes the apparatus to perform at least some of the functionalities according to any one of the embodiments described above.

As used in this application, the term ‘circuitry’ refers to all of the following: (a) hardware-only circuit implementations, such as implementations in only analog and/or digital circuitry, and (b) combinations of circuits and software (and/or firmware), such as (as applicable): (i) a combination of processor(s) or (ii) portions of processor(s)/software including digital signal processor(s), software, and memory(ies) that work together to cause an apparatus to perform various functions, and (c) circuits, such as a microprocessor(s) or a portion of a microprocessor(s), that require software or firmware for operation, even if the software or firmware is not physically present. This definition of ‘circuitry’ applies to all uses of this term in this application. As a further example, as used in this application, the term ‘circuitry’ would also cover an implementation of merely a processor (or multiple processors) or a portion of a processor and its (or their) accompanying software and/or firmware.

In an embodiment, at least some of the processes described in connection with above figures may be carried out by an apparatus comprising corresponding means for carrying out at least some of the described processes. Some example means for carrying out the processes may include at least one of the following: detector, processor (including dual-core and multiple-core processors), digital signal processor, controller, receiver, transmitter, encoder, decoder, memory, RAM, ROM, software, firmware, display, user interface, display circuitry, user interface circuitry, user interface software, display software, circuit, antenna, antenna circuitry, and circuitry. In an embodiment, the at least one processor, the memory, and the computer program code form processing means or comprises one or more computer program code portions for carrying out one or more operations according to any one of the described embodiments.

The techniques and methods described herein may be implemented by various means. For example, these techniques may be implemented in hardware (one or more devices), firmware (one or more devices), software (one or more modules), or combinations thereof. For a hardware implementation, the apparatus(es) of embodiments may be implemented within one or more application-specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described herein, or a combination thereof. For firmware or software, the implementation can be carried out through modules of at least one chip set (e.g. procedures, functions, and so on) that perform the functions described herein. The software codes may be stored in a memory unit and executed by processors. The memory unit may be implemented within the processor or externally to the processor. In the latter case, it can be communicatively coupled to the processor via various means, as is known in the art. Additionally, the components of the systems described herein may be rearranged and/or complemented by additional components in order to facilitate the achievements of the various aspects, etc., described with regard thereto, and they are not limited to the precise configurations set forth in the given figures, as will be appreciated by one skilled in the art.

Embodiments as described may also be carried out at least partly in the form of a computer process defined by a computer program or portions thereof. Embodiments of the methods described above may be carried out by executing at least one portion of a computer program comprising corresponding instructions. The computer program may be in source code form, object code form, or in some intermediate form, and it may be stored in some sort of carrier, which may be any entity or device capable of carrying the program. For example, the computer program may be stored on a computer program distribution medium readable by a computer or a processor. The computer program medium may be, for example but not limited to, a record medium, computer memory, read-only memory, electrical carrier signal, telecommunications signal, and software distribution package, for example. The computer program medium may be a non-transitory medium. Coding of software for carrying out the embodiments as shown and described is well within the scope of a person of ordinary skill in the art.

Even though the invention has been described above with reference to an example according to the accompanying drawings, it is clear that the invention is not restricted thereto but can be modified in several ways within the scope of the appended claims. Therefore, all words and expressions should be interpreted broadly and they are intended to illustrate, not to restrict, the embodiment.

1. An apparatus for determining an indication of pain, the apparatus comprising
at least one processor;
at least one memory including computer program code;
the at least one memory and the computer program code configured to, with the at least one processor, cause the apparatus at least to perform operations comprising:
obtaining video data stream of a subject;
obtaining audio data stream of the subject;
obtaining one or more biosignal data stream of the subject;
the video, audio and biosignal data being synchronized with each other and related to an event of care of the subject, and covering a given first time window before, during and after the event of care;
selecting a set of time points around the event of care and within the first time window;
selecting a second time window shorter than the first me window; select (210) video, audio and biosignal data stream at the set of time points, the length of the data stream being the duration of the second time window;
controlling an interface to play selected video and audio data streams;
obtaining using the interface from a plurality of user inputs describing estimates of pain experienced by the subject at the set of time points;
performing averaging of the obtained inputs;
based on the inputs, determining a correlation between the estimated pain experienced by the subject and the audio and biosignal data streams; and
storing the determined correlation as calibration parameters.

2. The apparatus of claim 1, wherein the at least one memory and the computer program code are configured to, with the at least one processor, cause the apparatus further to perform further operations comprising:
scaling the selected audio and biosignal data streams at the set of time points to a given scale; and
utilizing the scaled data streams when determining the correlation.

3. The apparatus of claim 2, wherein the at least one memory and the computer program code are configured to, with the at least one processor, cause the apparatus further to perform further operations comprising:
calculating an estimate for the pain experienced by a subject based on the determined correlation between the obtained input and the scaled data streams.

4. The apparatus of claim 1, wherein the at least one memory and the computer program code are configured to, with the at least one processor, cause the apparatus further to perform further operations comprising:
determining an average value of the inputs given by each user of the plurality of users;
calculating a scaling factor for the input given by each user of the plurality of users based on the determined average value; and
scaling the input of given by each user of the plurality of users with the scaling factor.

5. The apparatus of claim 1, wherein the biosignals comprise at least one of: Heart Rate Variability (HRV), heart rate, respiration rate blood oxygen level, body temperature, blood pressure, and skin impedance.

6. The apparatus of claim 1, wherein the at least one memory and the computer program code are configured to, with the at least one processor, cause the apparatus further to perform further operations comprising:
processing video stream data to obtain biosignal data from the video data stream.

7. The apparatus of claim 1, wherein the apparatus is a user terminal.

8. A system for determining an indication of pain, the system comprising:
an audio sensor for obtaining audio data stream of a subject;
a video capturing device for obtaining video data stream of a subject;
a biosensor for measuring one or more biosignals of a subject;
the video, audio and biosignal data being synchronized with each other and related to an event of care of the subject, and covering a given first time window before, during and after the event of care
a user interface and
a processing unit communicatively coupled with the audio sensor, video capturing device biosensor, the processing unit configured to perform operations comprising:
selecting a set of time points around the event of care and within the first time window;
selecting a second time window shorter than the first time window;
selecting video, audio and biosignal data stream at the set of time points, the length of the data stream being the duration of the second time window;
controlling an interface to play selected video and audio data streams;
obtaining using the interface from a plurality of user inputs describing estimates of pain experienced by the subject at the set of time points;
performing averaging of the obtained inputs;
based on the inputs, determining a correlation between the estimated pain experienced by the subject and the audio and biosignal data streams; and
storing the determined correlation as calibration parameters.

9. A method of determining an indication of pain, the method comprising:
obtaining video data stream of a subject;
obtaining audio data stream of the subject;
obtaining one or more biosignal data stream of the subject; the video, audio and biosignal data being synchronised with each other and related to an event of care of the subject, and covering a given first time window before, during and after the event of care;
selecting a set of points around the event of care and within the first time window;
selecting a second time window shorter than the e window;
selecting video, audio and biosignal data stream at the set of time points, the length of the data stream being the duration of the second time window;
controlling interface to play selected video and audio data streams;
obtaining using an interface from a plurality of user inputs describing estimates of pain experienced by the subject at the set of time points;
performing averaging of the obtained inputs;
based on the inputs, determining a correlation between the estimated pain experienced by the subject and the audio and biosignal data streams; and
storing the determined correlation as calibration parameters.

10. The method of claim 9, further comprising:
scaling the selected audio and biosignal data streams at the set of time points to a given scale; and
utilizing the scaled data streams when determining the correlation.

11. The method of claim 10, further comprising:
calculating an estimate for the pain experienced by a subject based on the determined correlation between the obtained input and the scaled data streams.

12. The method of claim 9, further comprising:
determining an average value of the inputs given by each user of the plurality of users; and
calculating a scaling factor for e input given by each user of the plurality of users based on the determined average value; and
scaling the input of given by each user of the plurality of users the scaling factor.

13. The method of claim 9, wherein the biosignals comprise at least one of: Heart Rate Variability (HRV), heart rate, respiration rate, blood oxygen level, body temperature, blood pressure, skin impedance.

14. The method of claim 9, further comprising processing video stream data to obtain biosignal data from the video data stream.
